(19) United States
(12) Patent Application Publication
(10) Pub. No.: US 2012/0052112 A1
(43) Pub. Date: Mar. 1, 2012

(54) RISPERIDONE-CONTAINING TRANSDERMAL PREPARATION AND ADHESIVE PATCH USING SAME

(75) Inventors: Mitsuru Kuribayashi, Ibaraki (JP); Motohiro Suzuki, Ibaraki (JP); Hidenao Fukushima, Tokyo (JP); Eisuke Shimizu, Tokyo (JP)

(73) Assignee: HISAMITSU PHARMACEUTICAL CO., INC., Tosu-shi, Saga (JP)

(21) Appl. No.: 13/202,465

(22) PCT Filed: Feb. 19, 2010
(86) PCT No.: PCT/JP2010/052541

§ 371 Date: Oct. 31, 2011

(30) Foreign Application Priority Data
Feb. 24, 2009 (JP) 2009-040918

(51) Int. Cl. A61K 9/70; C07D 471/04

(52) U.S. Cl. 424/449; 544/282

(57) ABSTRACT

A transdermal preparation is provided that contains risperidone and/or a pharmaceutically acceptable salt thereof as a drug, and the amount of the drug is 1 to 20 parts by mass based on 100 parts by mass of the total mass of the preparation. The skin permeation rate of the preparation is 0.5 to 30 μg/cm²/hour, and the drug diffusion coefficient of the preparation in the skin is 1.2×10⁻⁶ to 10.0×10⁻⁶ cm/hour.

### TECHNICAL FIELD

The present invention relates to a transdermal preparation containing risperidone and/or a pharmaceutically acceptable salt thereof as a drug, and an adhesive patch using the same.

### BACKGROUND ART

Risperidone is a benzisoxazole derivative compound developed by Janssen Pharmaceuticals, Inc (Belgium) (see Patent Literature 1). An anti-dopamin action, an anti-serotonin action, and a catalepsy-inducing action have been identified as its pharmacological actions, and widely used as a therapeutic drug for schizophrenia in clinical settings at present. Risperidone has been proposed to be applied to the treatment for hyperorexia and cosmetic compositions for the treatment of sensitive skin (see Patent Literatures 2 and 3).

Risperidone is believed to exert its effect on schizophrenia by regulation of the central nervous system mainly based on a dopamin D₂ receptor antagonistic action and a serotonin 5-HT₂ receptor antagonistic action. Risperidone is also known to have excellent effects not only on positive symptoms such as hallucination and obsession but also on negative symptoms such as emotional withdrawal and hypoesthesia. Risperidone also has a feature that side effects on the extrapyramidal system (tremor, stiffness and the like) are relatively lower than those in conventional antipsychotic drugs. Thus, risperidone is believed to be extremely useful for improving the QOL (quality of life) of patients.

Risperidone has been conventionally orally administered using tablets, granules or oral liquids. However, oral administration has drawbacks, e.g., the drug after being absorbed undergoes a first pass effect in the liver, and an unnecessarily high plasma concentration is temporarily detected after administration. Many side effects such as gastrointestinal disturbance, feeling of emesis and anorexia are reported in oral administration. Further, many patients with schizophrenia find it difficult to take regularly as an oral agent, and it has been described that its rate reaches about 75% of patients.

In order to solve the problems of oral administration as described above, methods of administration of risperidone through human skin has been investigated. For example, a medical patch containing risperidone and a skin permeation enhancer has been described in Patent Literature 4. Advantages such as reduced frequency of administration, enhancement of compliance, and easy administration and discontinuation are expected for administration using the adhesive patch.

### CITATION LIST

### Patent Literature

Patent Literature 1: Japanese Examined Patent Application Publication No. Hei-6-13511
Patent Literature 2: Japanese Unexamined Patent Application Publication (Translation of PCT Application) No. 2003-525865
Patent Literature 3: Japanese Unexamined Patent Application Publication (Translation of PCT Application) No. 2001-511782
Patent Literature 4: Japanese Unexamined Patent Application Publication (Translation of PCT Application) No. Hei-11-503138

### SUMMARY OF INVENTION

### Technical Problem

A horny layer in the skin, in which cells abundantly containing keratinous substances and intercellular lipids are laminated in layers, has a barrier function to prevent foreign matter from invading into the body. Thus, it is not easy to make transdermal absorbability of risperidone sufficiently excellent. This is considered to be one of the reasons why a risperidone-containing transdermal preparation is not yet in practical use. Even when the preparation excellent in transdermal absorbability is prepared and brought into contact with the skin, it takes a long time to obtain drug efficacy. Thus, there is room to improve for applying the conventional preparation to treatment.

The present invention has been made in view of the above circumstances, and it is an object of the present invention to provide a transdermal preparation capable of accomplishing the transdermal absorbability of risperidone as well as exerting its drug efficacy sufficiently and stably, and an adhesive patch using the same.

### Solution to Problem

The present inventors studied preparations excellent in transdermal absorbability of risperidone, and obtained a finding that unlike conventional drugs, even when a skin permeation rate is sufficiently increased by controlling a content of risperidone and selecting additives, the drug efficacy of risperidone is not exerted stably in some cases. As a result of further studying based on this finding, the present inventors have found that it is extremely efficient for stably exerting the drug efficacy to control not only the above skin permeation rate but also a drug diffusion coefficient in the skin, and completed the present invention.

The transdermal preparation according to the present invention contains risperidone and/or a pharmaceutically acceptable salt thereof as a drug. The above drug is contained in an amount of 1 to 20 parts by mass based on a total mass of 100 parts by mass of the preparation, the skin permeation rate is 0.5 to 30 μg/cm²/hour, and the skin diffusion coefficient in the skin is 1.2×10⁻⁶ to 10.0×10⁻⁶ cm/hour.

According to the above transdermal preparation, a plasma concentration of risperidone is more slowly increased after administration compared with an oral agent, and the drug efficacy is subsequently kept over a long period of time. Thus, the plasma concentration of risperidone (including a metabolite thereof) can be kept stably in the range suitable for exerting the drug efficacy. It has been described that risperidone exhibits the drug efficacy when the total plasma concentration of risperidone and the metabolite thereof is 20 to 50 ng/mL or a rate of the D₂ receptor occupied with risperidone is 65 to 80% (see Am Psychiatry 163: 3, March, 2006).

It is preferred that a ratio of the highest plasma concentration Cmaxto the lowest plasma concentration Cmin of the Sum of risperidone and the metabolite thereof (Cmax/Cmin) is constantly 5 or less when the above preparation is continuously brought into contact with the skin at a frequency of once a day to once 7 days. By setting the ratio of Cmax/Cmin to 5 or less in a steady state upon continuous administration, it is possible to accomplish both exertion of the drug efficacy and inhibition of the side effects at higher levels.

The risperidone-containing transdermal adhesive patch according to the present invention comprises a support, and a drug-containing layer formed on at least one surface of the support, and the drug-containing layer is composed of the above transdermal preparation of the present invention. By applying the above transdermal preparation to the adhesive patch, the high transdermal absorbability is accomplished still more stably. The adhesive patch has the advantage that administration to the patient is easier compared with oral agents and embrocation.

In the above adhesive patch, an area of the drug-containing layer to be contacted with the skin is preferably 5 to 100 cm². A thickness of the drug-containing layer is preferably 50 to 200 μm.

### Advantageous Effects of Invention

According to the present invention, excellent transdermal absorbability of risperidone can be accomplished, and drug efficacy can be exerted sufficiently and stably.

### BRIEF DESCRIPTION OF DRAWINGS

FIG. 1 is a view showing one example of a drug distribution in a drug-containing layer of the adhesive patch according to the present invention;

FIG. 2 is a graph showing results of human skin permeability tests (change of skin permeation rates);

FIG. 3 is a graph showing results of a comparative test of human skin permeability tests (change of skin permeation rates);

FIG. 4 is a graph showing a change of plasma concentrations of an active moiety after administration of an oral agent;

FIG. 5 is a graph showing a change of plasma concentrations of an active moiety after administration of an adhesive patch;

FIG. 6 is a graph showing changes of plasma concentrations of the active moiety in Case 1 and Comparative Case 1;

FIG. 7 is a graph showing changes of plasma concentrations of the active moiety in Case 2 and Comparative Case 2; and

FIG. 8 is a graph showing the change of the plasma concentration of the active moiety in Case 3.

### DESCRIPTION OF EMBODIMENTS

The risperidone-containing transdermal preparation according to the present invention will be described using the case of applying this to adhesive patches such as plaster agents and polutice as examples. The adhesive patch is a particularly preferable form in terms of drug absorbability and administration easiness. The preparation according to the present invention may be applied to cream, plasters, lotion, ointments, spray, and the like as long as the drug is absorbed through the skin.

(Transdermal Adhesive Patch)

The adhesive patch according to the present embodiment comprises a support, and a drug-containing layer formed on at least one surface of the support. The drug-containing layer is composed of the transdermal preparation containing risperidone and/or a pharmaceutically acceptable salt thereof as the drug.

The pharmaceutically acceptable salt of risperidone is not particularly limited, and includes, for example, inorganic salts such as hydrochloride, sulfate, nitrate, phosphate and hydrobromide salts, and organic salts such as acetate, propionate, citrate, lactate, oxalate, succinate, tartrate, malonate, fumarate and malate salts. Among them, the oxalate, hydrobromide and hydrochloride salts are preferable, and the oxalate salt is more preferable in terms of stability of the drug efficacy component in the preparation. The drug-containing layer may contain risperidone and the pharmaceutically acceptable salts alone or in mixture.

A content of totaled risperidone and the pharmaceutically acceptable salt thereof (hereinafter referred to as a “drug content”) is 1 to 20 parts by mass and preferably 2 to 15 parts by mass based on 100 parts by mass of the preparation that forms the drug-containing layer. When the drug content is less than one part by mass, it is difficult to make the plasma concentration of the sum of risperidone and the metabolite thereof (hereinafter simply referred to as a “plasma concentration”) 20 ng/mL or more in a steady state upon continuous administration, and the drug efficacy is not exerted sufficiently. On the other hand, when the drug content exceeds 20 parts by mass, the plasma concentration easily exceeds 50 ng/mL, and the occurrence of side effects is potentially increased.

The transdermal preparation having the drug-containing layer is prepared so that the skin permeation rate of the drug is 0.5 to 30 μg/cm²/hour. The skin permeation rate can be adjusted to a desired value by appropriately setting the content of risperidone and the additives (particularly an absorption enhancer) or the types of the additives. The “skin permeation rate” referred to here means a value calculated according to a formula (1):

Skin permeation rate=Pm×ΔC (1),

wherein Pm denotes a skin permeation coefficient of the drug, and ΔC denotes a difference in CG concentrations between a donor phase and a receptor phase.

When the skin permeation rate of the drug is less than 0.5 μg/cm²/hour, it is difficult to make the drug concentration in plasma to 20 ng/mL or more, and the drug efficacy of risperidone is not exerted sufficiently. On the other hand, when the skin permeation rate of the drug exceeds 30 μg/cm²/hour, the drug concentration in plasma is easily increased rapidly when compared with the case of 30 μg/cm²/hour or less, and consequently the side effects on the central nervous system characteristic of schizophrenia tends to occur. The skin permeation rate of the drug is preferably 1 to 25 μg/cm²/hour and more preferably 2 to 20 μg/cm²/hour.

The transdermal preparation having the drug-containing layer is prepared so that the drug diffusion coefficient in the skin is 1.2×10⁻⁶ to 10.0×10⁻⁶ cm/hour. The “drug diffusion coefficient in the skin” referred to here means a value calculated from measured values of the thickness of the horny layer and a time period (lag time) required for passing the drug through the horny layer (see R. J. Scheuplein, “mechanism of percutaneous absorption II. Transient diffusion and the relative importance of various routes of skin penetration,” J. Invest. Dermatol., 8, 79, 1967).

When the drug diffusion coefficient in the skin is less than 1.2×10⁻⁶ cm/hour, the drug concentration in plasma is not rapidly increased when compared with the case of 1.2×10⁻⁶ cm/hour or more, and consequently, the exertion of the drug efficacy tends to delay. On the other hand, when the drug diffusion coefficient in the skin exceeds 10.0×10⁻⁶ cm/hour, the drug concentration in plasma is easily increased rapidly when compared with the case of 10×10⁻⁶ cm/hour or less, and consequently the side effects on the central nervous system characteristic of schizophrenia tends to occur. The drug diffusion coefficient in the skin is preferably 1.2×10⁻⁶ to 9.0×10⁻⁶ cm/hour and more preferably 1.5×10⁻⁶ to 9.0×10⁻⁶ cm/hour.

Risperidone has a property of having a longer lag time compared with other drugs. The lag time of risperidone can be adjusted within the desired values by appropriately setting the content of risperidone and the additives (particularly a solubilizing agent, the absorption enhancer), or the types of the additives. According to the study by the present inventors, the lag time of risperidone can be shortened effectively by using acetic acid, propionic acid, lactic acid, sodium acetate, salicylic acid, benzoic acid, N-methyl-2-pyrrolidone, propylene glycol, or dipropylene glycol as the solubilizing agent for risperidone or using laurate diethanolamide, capric acid, isopropyl myristate, or monolaurate propylene glycol as the absorption enhancer. As the lag time is shortened, the drug diffusion coefficient in the skin becomes a large value.

The absorption enhancer is not particularly limited as long as it is a conventional compound having an absorption acceleration effect on the skin, and includes, for example, C₆₋₂₀ aliphatic alcohol, C₆₋₂₀ aliphatic ether, C₆₋₂₀ fatty acids, C₆₋₂₀ fatty acid ester, C₆₋₂₀ fatty acid amide, glycerine, glycerine fatty acid esters, propylene glycols, propylene glycol fatty acid esters, polyethylene glycol and polyethylene glycol fatty acid esters, aromatic organic acids, aromatic alcohol, aromatic organic acid ester, aromatic organic ether (the above compounds may be saturated or unsaturated, may be linear or branched, and may include a cyclic structure), lactate esters, acetate esters, monoterpene-base compounds, sesquiterpene, Azone, Azone derivatives, pyrothiodecane, sorbitan fatty acid esters (Span-based), polysorbate-based (Tween-based), polyoxyethylene cured castor oil-based (HCO-based), polyoxyethylene alkyl ethers, sucrose fatty acid esters, and vegetable oils.

Specific examples of the absorption enhancer include caprylic acid, capric acid, caproic acid, lauric acid, myristic acid, palmitic acid, stearic acid, isostearic acid, oleic acid, linoleic acid, linolenic acid, lauryl alcohol, myristic alcohol, oleic alcohol, isostearic alcohol, cetyl alcohol, methyl laurate, hexyl laurate, diethanolamide laurate, isopropyl myristate, myristyl myristate, octyldodecyl myristate, cetyl palmitate, salicylic acid, methyl salicylate, salicylate ethylene glycol, cinnamic acid, methyl cinnamate, cresol, cetyl lactate, lauryl lactate, ethyl acetate, propyl acetate, geraniol, thymol, eugenol, terpineol, l-menthol, borneorol, d-limonene, isoeugenol, isoborneol, nerol, dl-camphor, glycerine monocaprylate, glycerine monocaprate, glycerine monolaurate, glycerine monooleate, sorbitan monolaurate, sucrose monolaurate, polysorbate 20, propylene glycol, propylene glycol monolaurate, polyethylene glycol monolaurate, polyethylene glycol monostearate, polyoxyethylene lauryl ether, HCO-60, pyrothiodecane, olive oil and sorbitan monooleate.

The above absorption enhancer can be used alone or in mixture of two or more. The content of the absorption enhancer is preferably 1 to 20 parts by mass, more preferably 2 to 15 parts by mass and still more preferably 3 to 10 parts by mass based on 100 parts by mass of the preparation that forms the drug-containing layer. By setting the content of the absorption enhancer within the above, the skin permeability of the drug is improved compared with the case out of the above range, and irritation to the skin, such as rashes and edema is reduced.

The drug-containing layer preferably further contains a pressure-sensitive adhesive base, a plasticizer and a tackifier.

The pressure-sensitive adhesive base is not particularly limited as long as it is a compound having adhesiveness, and includes, for example, thermoplastic elastomers, acrylic polymers, rubber-based polymers, polyurethane-based polymers, and hydrophobic polymers such as polydimethylsiloxane.

The acrylic polymer is not particularly limited as long as it is a copolymer obtained by containing at least one (meth)acrylate derivative, and is preferably an acrylate ester copolymer. Specific examples of the acrylate ester copolymer include copolymers of at least two selected from the group consisting of 2-ethylhexyl acrylate, vinyl acetate, methacrylate, methoxyethyl acrylate, hydroxyethyl acrylate and acrylic acid.

Specific examples of the acrylic polymer include acrylic acid/octyl acrylate ester copolymers, 2-ethylhexyl acrylate/vinyl pyrrolidone copolymer solutions, acrylate ester/vinyl acetate copolymers, 2-ethylhexyl acrylate/2-ethylhexyl methacrylate/dodecyl methacrylate copolymers, methyl acrylate/2-ethylhexyl acrylate copolymer resin emulsion, acrylic polymers contained in acryl resin alkanol amine solutions, DURO-TAK acryl adhesive series (supplied from national Starch and Chemical Company), and Eudragit series (HIGUCHI Inc.) which are listed as the adhesives in the Pharmaceutical Excipient Dictionary 2000 (edited by International Pharmaceutical Excipients Council Japan).

The rubber-based polymers are not particularly limited, and include, for example, styrene-isoprene-styrene block copolymers (hereinafter, also referred to as “SIS”), isoprene rubbers, polyisobutylene (hereinafter, referred to as “PIB”), styrene-butadiene-styrene block copolymers (hereinafter, referred to as “SBS”), styrene-butadiene rubbers (hereinafter, referred to as “SBR”), and polysiloxane. SIS and the acrylate ester copolymer compounds are particularly preferable among the aforementioned compounds.

Such a pressure-sensitive adhesive base can be used alone or in mixture of two or more. The content of the pressure-sensitive adhesive base is preferably 5 to 50 parts by mass, more preferably 10 to 40 parts by mass and still more preferably 10 to 30 parts by mass based on 100 parts by mass of the preparation that forms the drug-containing layer. By setting the content of the pressure-sensitive adhesive base within the above range, the stability of the formed drug-containing layer and the skin permeability of the drug are enhanced when compared with the case out of the above range.

The plasticizer is not particularly limited as long as it is a compound having plasticity, and includes, for example, petroleum-based oils (e.g., paraffin-based process oils, naphthene-based process oils, aromatic process oils, and the like), squalane, squalene, vegetable oils (e.g., olive oil, camellia oil, castor oil, toll oil, peanut oil), silicon oil, dibasic acid ester (e.g., dibutyl phthalate, dioctyl phthalate, and the like), liquid rubbers (e.g., polybutene, liquid isoprene rubber), liquid fatty acid esters (isopropyl myristate, hexyl laurate, diethyl sebacate, diisopropyl sebacate), diethylene glycol, polyethylene glycol, salicylic acid glycol, propylene glycol, dipropylene glycol, triacetin, triethyl citrate, and crotamiton. Liquid paraffin, liquid polybutene, salicylic acid glycol and crotamiton are preferable among them.

Such a plasticizer can be used alone or in mixture of two or more. The content of the plasticizer is preferably 5 to 30 parts by mass, more preferably 10 to 30 parts by mass and still more preferably 10 to 20 parts by mass based on 100 parts by mass of the preparation that forms the drug-containing layer. By setting the content of the plasticizer within the above range, the skin permeability of the drug is enhanced, and a cohesive force as the adhesive patch is enhanced when compared with the case out of the above range.

When an adhesive force of the drug-containing layer is insufficient, it is preferable to add a tackifier. The tackifier is not particularly limited, and includes, for example, rosin derivatives (e.g., rosin, glycerine ester of rosin, hydrogenated rosin, glycerine ester of hydrogenated rosin, pentaerythritol ester of rosin, and the like), alicyclic saturated hydrocarbon resins (e.g., Alcon P100 supplied from Arakawa Chemical Industries Ltd.), aliphatic hydrocarbon resins (e.g., Quinton B170 supplied form Nippon Zeon Co., Ltd.), terpene resins (e.g., Clearon P-125 supplied from Yasuhara Chemical Co., Ltd.), and maleic acid resins. Glycerine ester of hydrogenated rosin, the alicyclic hydrocarbon resin, the aliphatic hydrocarbon resin, and the terpene resin are preferable among them.

Such a tackifier can be used alone or in mixture of two or more. The content of the tackifier is preferably 20 to 60 parts by mass, more preferably 30 to 60 parts by mass and still more preferably 35 to 60 parts by mass based on 100 parts by mass of the preparation that forms the drug-containing layer. When the content of the tackifier is less than 20 parts by mass, the adhesive force as the adhesive patch tends to be reduced when compared with the case of the above range. Also when the content of the tackifier exceeds 60 parts by mass, the irritation to the skin upon being peeled tends to become strong when compared with the case of the above range.

The drug-containing layer may contain an antioxidant, a filler, a crosslinking agent, a preservative, or an ultraviolet ray absorber, or the like if necessary. Tocopherol and ester derivatives thereof, ascorbic acid, ascorbate stearate ester, nordihydroguaiaretic acid, dibutylhydroxytoluene (BHT), butylhydroxyanisole, and the like are preferable as the antioxidant. Calcium carbonate, magnesium carbonate, silicate salts (e.g., aluminium silicate, magnesium silicate, and the like), silicic acid, barium sulfate, calcium sulfate, calcium zincate, zinc oxide, titanium oxide, and the like are preferable as the filler. Amino compounds, phenol compounds, epoxy compounds, isocyanate compounds, organic peroxides, metal alcholate, metal chelate, and the like are preferable as the crosslinking agent. Ethyl paraoxybenzoate, propyl paraoxybenzoate, butyl paraoxybenzoate, and the like are preferable as the preservative. p-Aminobenzoic acid derivatives, anthranic acid derivatives, salicylic acid derivatives, coumarin derivatives, amino acid-based compounds, imidazoline derivatives, pyrimidine derivatives, dioxane derivatives, and the like are preferable as the ultraviolet ray absorber.

A total amount of such an antioxidant, filler, crosslinking agent, preservative and ultraviolet ray absorber is preferably 10 parts by mass or less, more preferably 5 parts by mass or less and still more preferably 2 parts by mass or less based on 100 parts by mass of the preparation that forms the drug-containing layer.

The adhesive patch of the present embodiment can be produced by a conventional method such as a solvent method or a hot melt method. For example, when produced by the solvent method, the adhesive patch of the present embodiment can be obtained by adding other ingredients to an organic solvent solution of the composition to be combined, mixing the resulting solution followed by spreading it on the support and drying it to form the drug-containing layer. When the composition to be combined can be coated by the hot melt method, the adhesive patch of the present embodiment can also be obtained by dissolving the composition at high temperature and subsequently spreading it on the support to form the drug-containing layer.

The solvent used when producing by the solvent method includes, for example, compounds such as lower alcohol, toluene, ethyl acetate, hexane, cyclohexane and heptane used as the production solvents and the plasticizer for preparations. Methanol, ethanol, isopropanol, toluene, ethyl acetate, cyclohexane and heptane are preferable, and methanol, ethanol, toluene, heptane and ethyl acetate are particularly preferable among them.

The adhesive patch of the present embodiment can also be obtained by forming the drug-containing layer using a peeling liner described later in place of the support and then attaching the support.

As long as the adhesive patch of the present embodiment has the drug-containing layer composed of the above composition and has the support that supports it, the other layers and the components that compose them are not particularly limited. For example, the adhesive patch of the present embodiment can comprise the peeling liner provided on the drug-containing layer in addition to the support and the drug-containing layer.

The support is not particularly limited as long as it is suitable for supporting the drug-containing layer, and elasticized and non-elasticized ones can be used. Specific examples thereof include fabrics, nonwoven, polyurethane, polyester, polyvinyl acetate, polyvinylidene chloride, polyethylene, polyethylene terephthalate, aluminium sheets, and the like, and composites thereof.

The peeling liner is not particularly limited as long as it has a sufficient peel property from the drug-containing layer, and polyethylene terephthalate (PET) films, polyethylene films, polypropylene films, polytetrafluoroethylene films, polyvinyl chloride films, polyvinylidene chloride films, laminate films of high-quality paper with polyolefin, and the like can be used suitably. In order to enhance easiness of working upon peeling the peeling liner from a patch side, it is preferable to give a fluorine treatment or a silicon treatment to the surface on the side contacted with an adhesive layer of the peeling liner.

In the adhesive patch according to the present embodiment, an applied amount of the active drug can be easily controlled depending on symptoms, age, body weight, sex and the like of a patient by cutting the adhesive patch. An area of the drug-containing layer of the adhesive patch to be contacted with the skin is not particularly limited, and is preferably 5 to 100 cm², and more preferably 10 to 80 cm². By setting the area of the drug-containing layer of the adhesive patch to be contacted with the skin to 100 cm² or less, the patch is handled suitably upon being applied. By setting it to 5 cm² or more, it becomes easy to keep sufficient skin permeability of the drug.

A thickness of the drug-containing layer is preferably 50 to 200 μm and more preferably 70 to 170 μm. By setting the thickness of the drug-containing layer to 200 μm or less, the patch is handled suitably upon being applied. By setting it to 50 μm or more, it becomes possible to contain a sufficient amount of the drug in the drug-containing layer.

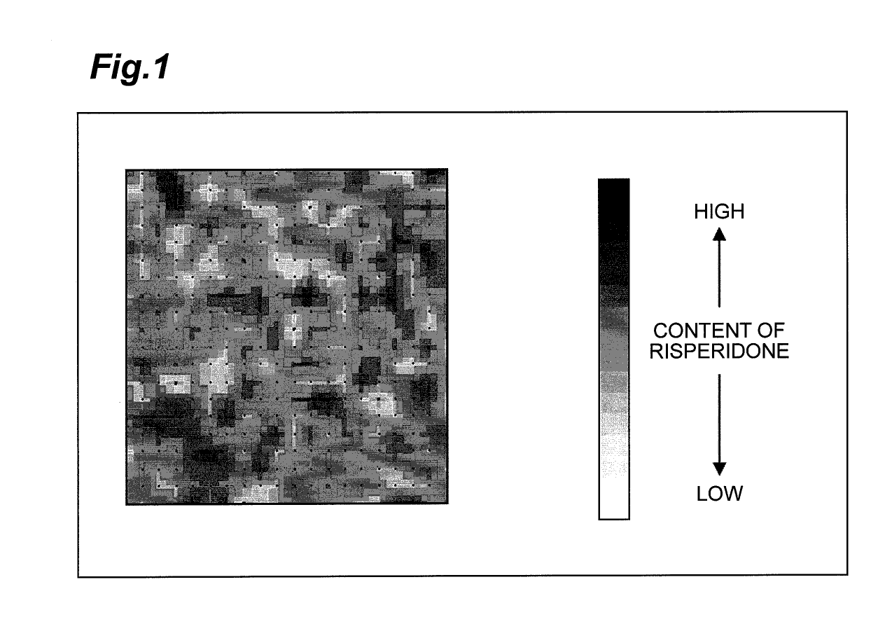

The drug is not required to always be uniformly dispersed in the drug-containing layer, and the drug may be distributed so as to form domains as shown in FIG. 1. In FIG. 1, the content of the drug in the adhesive patch according to the present embodiment was measured under the following condition, and the measured values were mapped:

Measurement method: Reflection method

Measured area: 2×2 mm

Aperture: 100×100 μm

Point number: 20×20 points

Mapping strength: 1684 cm⁻¹ (C═O stretching vibration of risperidone)

The drug prepared so that the content of the drug, the skin permeation rate and the drug diffusion coefficient in the skin are in the predetermined range, respectively as described above is used as the drug-containing layer in the adhesive patch according to the present embodiment. By virtue of such a constitution, administration of the adhesive patch can sufficiently and stably keep the total plasma concentration of risperidone and its metabolite in the range of 20 to 50 ng/mL.

It is preferred that the adhesive patch according to the present embodiment is continuously brought into contact with the skin at a frequency of once a day to once 7 days to constantly keep the ratio of Cmax/Cmin to 5 or less. Cmax means a maximum value of the total plasma concentration of risperidone and its metabolite, and Cmin means a minimum value of the total plasma concentration of risperidone and its metabolite. The ratio of Cmax/Cmin of an oral agent and a Depo preparation (subcutaneous) of risperidone commercially available at present exceeds 5. By setting the ratio of Cmax/Cmin to 5 or less, the potential for causing side effects can be sufficiently reduced, and the gap between a state in which the drug efficacy is obtained and a state in which the drug efficacy is not obtained becomes sufficiently small, and this is also suitable in terms of compliance of the patient.

When the ratio of Cmax/Cmin in a steady state upon being continuously administered is 5 or less, the plasma concentration can be in the range of 20 to 50 ng/mL at a mean value of multiple patients. When the plasma concentration is kept within this range, a rate of dopamine D₂ receptor occupied by risperidone can be 65 to 80%, and the drug efficacy is effectively exerted. That is, according to the adhesive patch according to the present embodiment, the side effects derived from risperidone can be reduced as much as possible because the rate of the dopamine D₂ receptor occupied by risperidone can be 80% or less.

(Method of Prolonging Drug Efficacy of Risperidone)

The above transdermal preparation exerts the effect of keeping the drug efficacy of risperidone over a long period of time. Therefore, the above transdermal preparation or the adhesive patch using the same provides a method of prolonging the drug efficacy of risperidone, comprising a step of bringing the transdermal preparation into contact with the skin. According to this method, the plasma concentration of risperidone can be increased more slowly and the side effects can be inhibited sufficiently after administration compared with oral agents.

(Method of Stabilizing Drug Efficacy of Risperidone)

The above transdermal preparation exerts the effect of stably eliciting the drug efficacy of risperidone. Therefore, the above transdermal preparation or the adhesive patch using the same provides a method of stabilizing the drug efficacy of risperidone, comprising a step of continuously bringing the transdermal preparation into contact with the skin at a frequency of once a day to once 7 days. According to this method, the ratio of Cmax/Cmin can be relatively easily controlled to 5 or less. This can accomplish both the exertion of the drug efficacy and the inhibition of the side effects at still higher levels.

### EXAMPLES

The present invention will be described below more specifically with reference to the following Examples and Comparative Examples, but the present invention is not limited thereto.

### Example 1

Risperidone, liquid paraffin, propylene glycol monolaurate, acetic acid and sodium acetate were mixed thoroughly. A mixed solution composed of a styrene-isoprene-styrene block copolymer (SIS), an alicyclic saturated hydrocarbon resin and toluene was added to the resulting mixture to prepare a coating solution for a drug-containing layer. This coating solution was spread onto a peeling liner, and the solvents were dried and removed to form the drug-containing layer. Further, a support was attached to the drug-containing layer to obtain an adhesive patch. A mass rate of each component was as shown in columns for Example 1 in Table 1. A thickness was 75 μm and a content of the drug was 0.75 mg/cm² in the drug-containing layer.

### Example 2

An adhesive patch was obtained in the same manner as in Example 1, except that a coating solution for a drug-containing layer having the mass rates shown in the columns for Example 2 in Table 1 was used. The thickness was 100 μm and the content of the drug was 1.0 mg/cm² in the drug-containing layer. The coating solution for the drug-containing layer was prepared as follows. That is, risperidone, liquid paraffin, propylene glycol monolaurate, sorbitan monolaurate, acetic acid and sodium acetate were mixed thoroughly. A mixed solution composed of the styrene-isoprene-styrene block copolymer (SIS), the alicyclic saturated hydrocarbon resin, an acrylate ester copolymer (DURO-TAK 87-2194) and toluene was added to the resulting mixture to prepare the coating solution for the drug-containing layer.

### Example 3

An adhesive patch was obtained in the same manner as in Example 1, except that a coating solution for the drug-containing layer having the mass rates shown in the columns for Example 3 in Table 1 was used. The thickness was 75 μm and the content of the drug was 0.75 mg/cm² in the drug-containing layer. The coating solution for the drug-containing layer was prepared as follows. That is, risperidone, liquid paraffin, propylene glycol monolaurate, capric acid, acetic acid and sodium acetate were mixed thoroughly. A mixed solution composed of the styrene-isoprene-styrene block copolymer (SIS), the alicyclic saturated hydrocarbon resin, an acrylate ester copolymer (DURO-TAK 87-2194) and toluene was added to the resulting mixture to prepare the coating solution for the drug-containing layer.

### Example 4

An adhesive patch was obtained in the same manner as in Example 1, except that a coating solution for the drug-containing layer having the mass rates shown in the columns for Example 4 in Table 1 was used. The thickness was 100 μm and the content of the drug was 0.8 mg/cm² in the drug-containing layer. The coating solution for the drug-containing layer was prepared as follows. That is, risperidone, liquid paraffin, propylene glycol monolaurate, acetic acid and sodium acetate were mixed thoroughly. A mixed solution composed of the styrene-isoprene-styrene block copolymer (SIS), the alicyclic saturated hydrocarbon resin, an acrylate ester copolymer (DURO-TAK 87-2516) and toluene was added to the resulting mixture to prepare the coating solution for the drug-containing layer.

### Comparative Example 1

An adhesive patch was obtained in the same manner as in Example 1, except that a coating solution for the drug-containing layer having the mass rates shown in the columns for Comparative Example 1 in Table 1 was used. The thickness was 120 μm and the content of the drug was 1.8 mg/cm² in the drug-containing layer. The coating solution for the drug-containing layer was prepared as follows. That is, risperidone, propylene glycol monolaurate, acetic acid and sodium acetate were mixed thoroughly. A mixed solution composed of the acrylate ester copolymer (DURO-TAK 87-2516) and polyvinyl pyrrolidone (K30) was added to the resulting mixture to prepare the coating solution for the drug-containing layer.

<Human Skin Permeability Test>

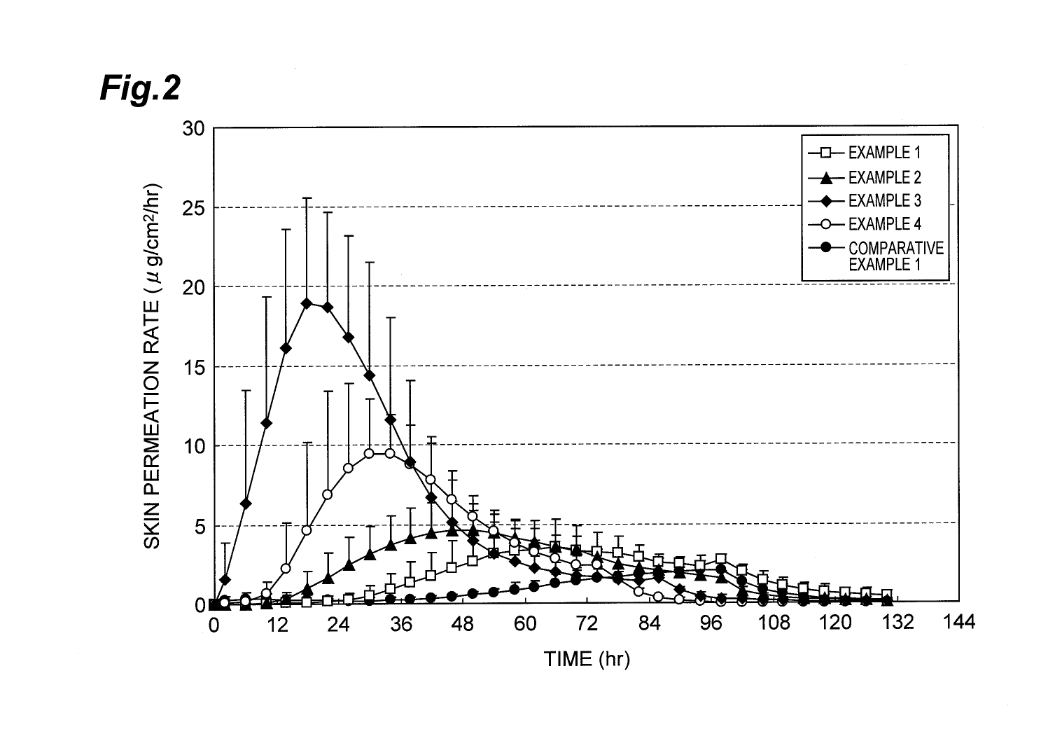

A human skin permeability test was performed using each adhesive patch according to Examples 1 to 4 and Comparative Example 1 as a test preparation. The test preparation (3 cm²) was put on a side of a horny layer of human skin isolated from a dead body and cut into a thickness of about 500 μm using a dermatome. The dermis side of the skin was directed to a side of a receptor layer, and the skin was loaded in a flow-through-cell warmed at 32° C. Saline was used as a receptor solution, and the receptor solution was supplied into the flow through cell at a constant flow rate (3 mL/hour). An aliquot of the receptor solution was sampled every 4 hours, and a drug concentration therein was measured by high performance liquid chromatography. A lag time, a skin permeation rate of the drug, and a drug diffusion coefficient in the skin were calculated from measured values of the drug concentrations and flow volumes measured precisely. Results are shown in Table 1 and FIG. 2.

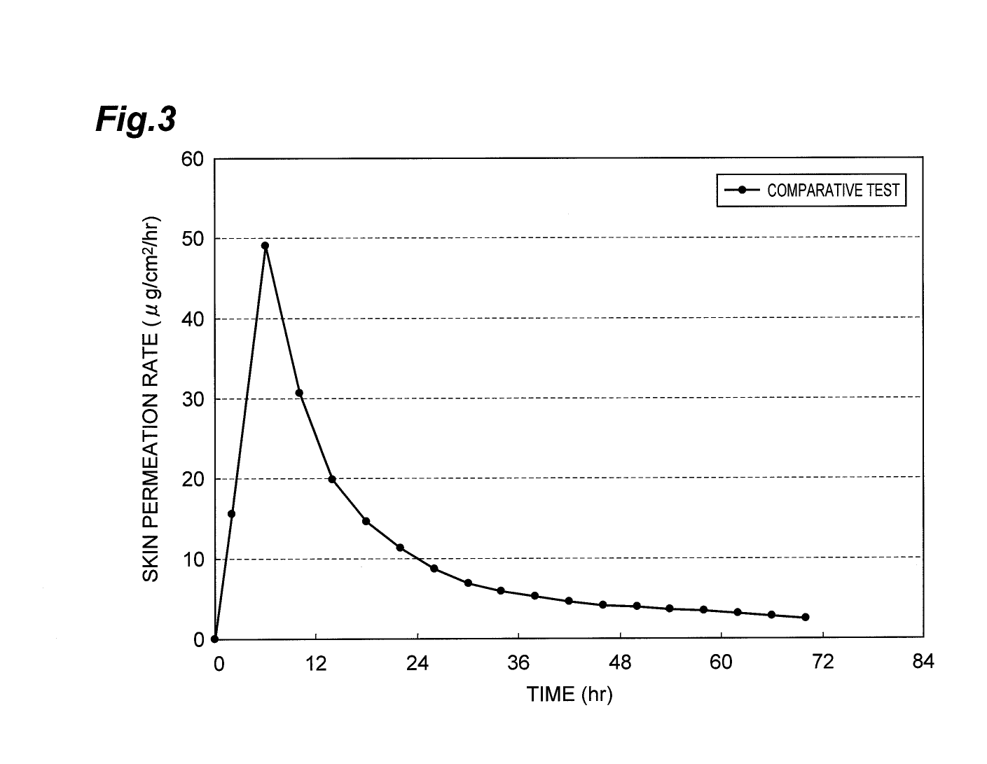

As a comparative test, the human skin permeability test was carried out under the same condition as above, except that the adhesive patch (3 cm²) according to Example 4 was adhered to the skin in which pores had been formed in the horny layer by a physical mean. As a result, the lag time, the maximum skin permeation rate and the drug diffusion coefficient in the skin were 2.73 hours, 48.99 μg/cm²/hour, and 24.5×10⁻⁶ cm/hour, respectively. The results are shown in FIG. 3.

TABLE 1

| | | | | | | Comparative |
|---|---|---|---|---|---|---|
| | | Example 1 | Example 2 | Example 3 | Example 4 | Example 1 |
| Drug | Risperidone | 10.0 | 10.0 | 10.0 | 8.0 | 15.0 |
| Absorption enhancer | Propylene glycol | 3.0 | 7.0 | 8.0 | 8.0 | 5.0 |
| | monolaurate | | | | | |
| | Sorbitan monolaurate | — | 0.5 | — | — | — |
| | Capric acid | — | — | 2.0 | — | — |
| Pressure-sensitive | SIS | 12.2 | 12.2 | 10.2 | 12.0 | — |
| adhesive base | Acrylate ester | — | 10.0 | 10.0 | — | — |
| | copolymer | | | | | |
| | (DURO-TAK | | | | | |
| | 87-2194) | | | | | |
| | Acrylate ester | — | — | — | 5.0 | 62.4 |
| | copolymer | | | | | |
| | (DURO-TAK | | | | | |
| | 87-2516) | | | | | |
| | Polyvinyl pyrrolidone | — | — | — | — | 5.0 |
| | (K30) | | | | | |
| Plasticizer | Liquid paraffin | 15.4 | 9.7 | 10.0 | 9.4 | — |
| Tackifier | Alicyclic saturated | 53.0 | 42.2 | 37.2 | 45.0 | — |
| | hydrocarbon resin | | | | | |
| Solubilizing agent | Acetic acid | 4.4 | 4.4 | 6.6 | 6.6 | 6.6 |
| | Sodium acetate | 2.0 | 4.0 | 6.0 | 6.0 | 6.0 |
| Evaluation | Lag time (hr) | 42.79 | 25.49 | 7.66 | 17.6 | 57.37 |
| | Highest skin | 3.53 | 4.61 | 18.94 | 9.41 | 1.90 |
| | permeation rate | | | | | |
| | (μg/cm²/hr) | | | | | |
| | Drug diffusion | 1.56 × 10⁻⁶ | 2.62 × 10⁻⁶ | 8.71 × 10⁻⁶ | 3.79 × 10⁻⁶ | 1.16 × 10⁻⁶ |
| | coefficient in skin | | | | | |
| | (cm/hr) | | | | | |

<Plasma Concentration Measurement Test>

A test in which the adhesive patch according to Example 4 was administered to healthy adult males, and measurement of the plasma concentration of the drug in the subject was carried out as follows. That is, a one-way crossover test in which first, an oral agent of risperidone (Risperdal 1 mg) was orally administered to multiple healthy adult males that composed Group 1, and after providing a certain cessation of the drug, the adhesive patch (content of risperidone: 4 mg, area: 5 cm²) according to Example 4 was adhered to the skin for over 24 hours was carried out. Changes of plasma concentrations of risperidone and 9-OH-risperidone that were a major metabolite of risperidone and pharmacokinetic parameters were studied.

The changes of the plasma concentrations and the pharmacokinetic parameters were studied in the same manner as above, except that the adhesive patch according to Example 4 was adhered to the skin over 72 hours instead of 24 hours in multiple healthy adult males that composed Group 2.

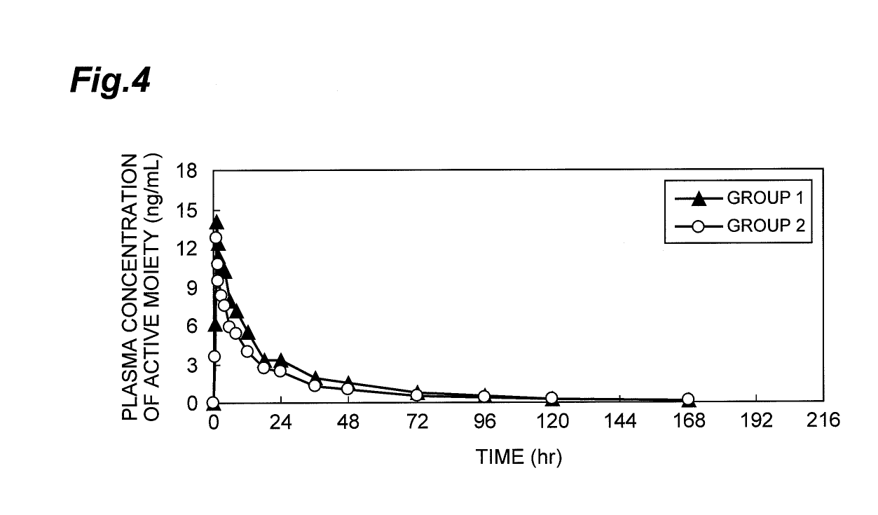

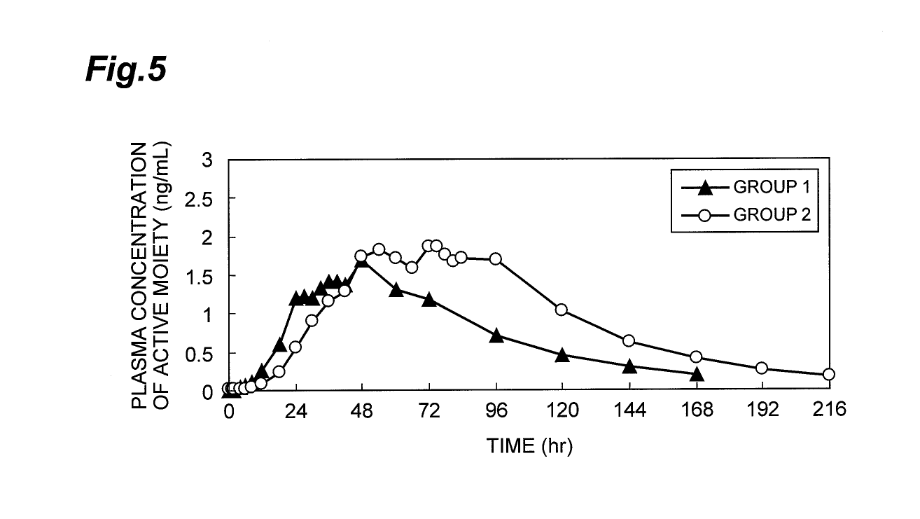

The changes of the plasma concentrations of active moiety (risperidone and 9-OH-risperidone) after administration of the oral agent of risperidone are shown in FIG. 4. The changes of the plasma concentrations of the active moiety (risperidone and 9-OH-risperidone) after administration of the adhesive patch according to Example 4 are shown in FIG. 5. As shown in FIG. 5, the plasma concentrations of the active moiety were gently increased when the adhesive patch according to Example 4 was adhered to the skin in both Groups 1 and 2. Values of Cmax, Tmax and t_{1/2} after the administration of the oral agent and the adhesive patch are shown in Table 2. In Table, Tmax means a time period until reaching the maximum of the plasma concentration after the start of the test, and t_{1/2} means a time period until lowering to a half maximum concentration in a terminal phase.

TABLE 2

| | | Application | | | |
|---|---|---|---|---|---|
| | | time | Cmax | Tmax | t_{1/2} |
| | | (hr) | (ng/mL) | (hr) | (hr) |
| Oral | Group 1 | 24 | 14.6 | 1.17 | 39.0 |
| agent | (N = 12) | | | | |
| | Group 2 | 72 | 12.7 | 1.08 | 40.3 |
| | (N = 12) | | | | |
| Adhesive | Group 1 | 24 | 1.99 | 50.0 | 41.2 |
| patch | (N = 12) | | | | |
| | Group 2 | 72 | 2.18 | 70.6 | 39.6 |
| | (N = 11) | | | | |

The lag time and the like were calculated by a deconvolution method from the plasma drug concentration in a single dose, which was obtained from the result of the above measurement. The lag time, mean value, minimum and maximum values of the highest drug permeation rate of the adhesive patch, and blood metabolite ratios (AUC ratios of 9-OH RIS/RIS) of the adhesive patch and the oral agent are shown in Table 3.

TABLE 3

| | | Group 1 | Group 2 |
|---|---|---|---|
| Lag time (hr) | Mean value | 21.3 | 25.6 |
| | Minimum value | 5.2 | 12.1 |
| | Maximum value | 37.8 | 44.3 |
| Highest drug | Mean value | 3.8 | 2.3 |
| permeation rate | Minimum value | 0.6 | 1.5 |
| (μg/cm²/hr) | Maximum value | 7.8 | 3.2 |
| Blood | Adhesive patch | 3.38 | 4.97 |
| metabolite ratio | Oral agent | 5.37 | 7.33 |
| (9-OH RIS/RIS, | | | |
| AUC) | | | |

As shown in FIGS. 4 and 5, a rising of the plasma concentration was slower in the adhesive patch than in the oral agent, and the change of the plasma concentration of prolactin was also slow in correlation with it. The plasma concentration of prolactin after administration of the oral agent was changed in the range of 3.1 to 106 ng/mL On the contrary, the plasma concentration of prolactin after administration of the adhesive patch was changed in the range of 2.1 to 37.3 ng/mL in Group 1 and 2.8 to 37.3 ng/mL in Group 2. The variation of the plasma concentration of prolactin was smaller when the adhesive patch was used than when the oral agent was used. It was also found that the metabolite ratio in plasma was smaller in the adhesive patch than in the oral agent.

<Study on Change of Plasma Drug Concentration>

### (Case 1)

The change of the plasma drug concentration was studied by an overlapping method wherein the adhesive patch (patch area: 20 cm²) according to Example 4 were repeatedly administrated in a manner that a patch adhered to the skin were changed to a new adhesive patch every 24 hours. A reason why the patch area was 20 cm² was that the patch area of the adhesive patch corresponding to administration of the oral agent containing 2 mg of risperidone was estimated to be 20 cm² when the adhesive patch was adhered over 24 hours by calculating an area under a curve of the graph showing the change of the plasma concentrations shown in FIG. 5.

(Case 2)

The change of the plasma drug concentration was studied by the same methodologies as in Case 1, when repeated administration in which the adhesive patch (patch area: 30 cm²) according to Example 4 was adhered to the skin for 72 hours and then the adhered adhesive patch was changed to a new adhesive patch was carried out.

(Comparative Case 1)

The change of the plasma drug concentration was studied by the same methodologies as in Case 1, when 1 mg per dose of risperidone was repeatedly administered by the oral agent divided twice daily.

(Comparative Case 2)

The change of the plasma drug concentration was studied by the same methodologies as in Case 2, when 1 mg per dose of risperidone was repeatedly administered by the oral agent divided twice daily.

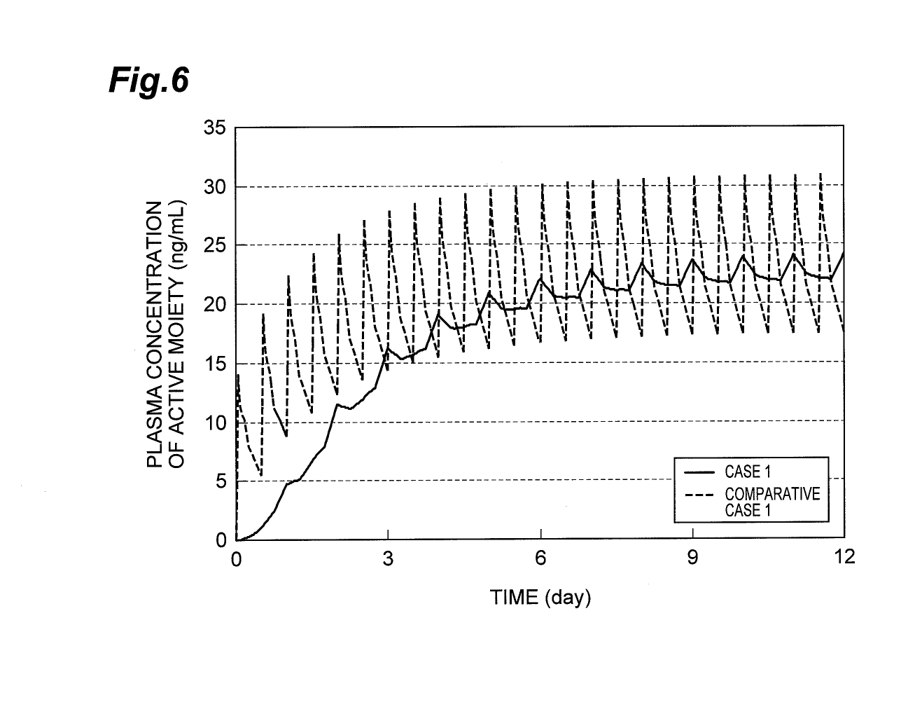

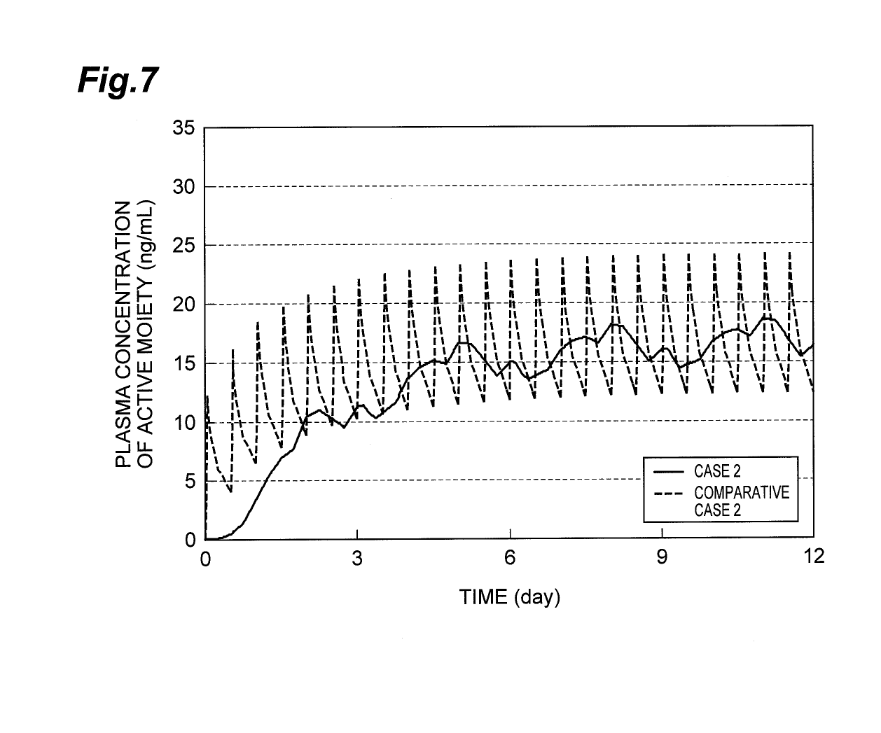

As shown in FIGS. 6 and 7, in both Case 1 and Case 2, it took about 6 days to reach a steady state from the start of administration. The ratio of Cmax/Cmin in the drug concentration in plasma was 1.03 to 1.25 in Case 1 (adhered for 24 hours) and 1.15 to 1.67 in Case 2 (adhered for 72 hours). In the oral agent, the ratio of Cmax/Cmin was 1.54 to 2.21 in Case 1 and 1.67 to 2.32 in Case 2. It was reported that the ratio of Cmax/Cmin of the preparation for intramuscular injection was 2.38 to 3.96. Thus, it was found that the ratio of Cmax/Cmin when the present adhesive patch was repeatedly adhered was significantly smaller when compared with the oral agent and the preparation for intramuscular injection.

(Case 3)

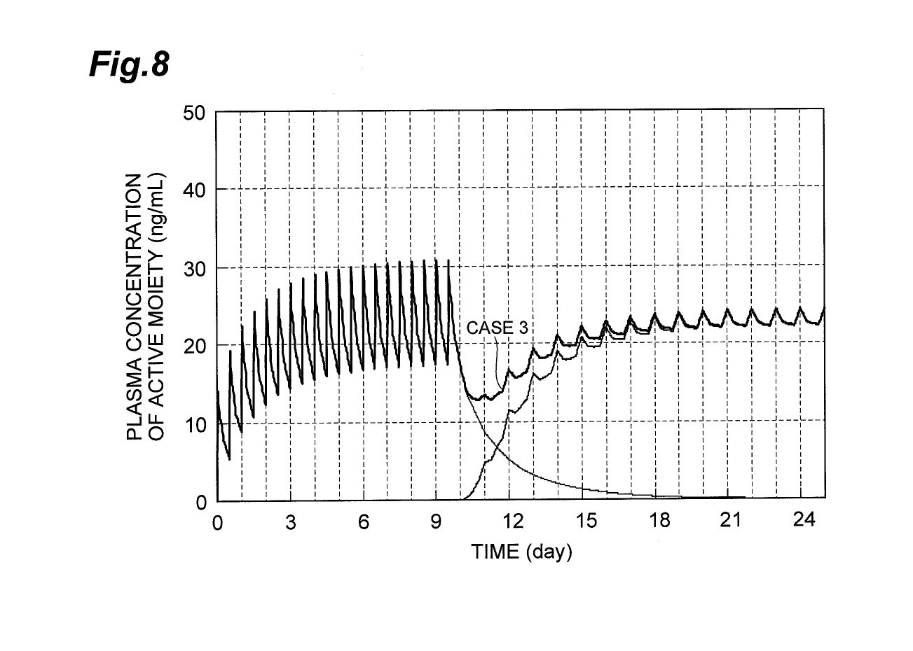

It has been reported that it is necessary to combine the oral agent with the preparation for intramuscular injection for about 3 weeks until the effective plasma concentration is accomplished because when the oral agent is switched to the preparation for intramuscular injection, latency of drug release is observed for about 2 weeks after starting administration of the preparation for intramuscular injection. A time period required for the combination thereof was studied when the oral agent was switched to the adhesive patch. The study was carried out based on the results of Case 1 and Comparative Case 1, and the effective plasma concentration could be kept without providing the combination period as shown in FIG. 8. It is predicted that the combination period can be shortened sufficiently when the oral agent is switched to the adhesive patch compared with when switched to the preparation for intramuscular injection.

### INDUSTRIAL APPLICABILITY

According to the present invention, excellent transdermal absorbability of risperidone can be accomplished, and it is possible to produce the drug efficacy sufficiently and stably.

1.-5. (canceled)

6. A risperidone-containing transdermal preparation comprising risperidone and/or a pharmaceutically acceptable salt thereof as a drug,
wherein the amount of the drug is 1 to 20 parts by mass based on 100 parts by mass of the total mass of the preparation, wherein a skin permeation rate is 0.5 to 30 μg/cm²/hour, wherein a drug diffusion coefficient in the skin is 1.2×10⁻⁶ to 10.0×10⁻⁶ cm/hour.

7. The preparation according to claim 6, wherein a ratio of a highest plasma concentration (Cmax) to a lowest plasma concentration (Cmin) of the sum of the risperidone and a metabolite thereof (Cmax/Cmin) is 5 or less in a steady state when the preparation is continuously brought into contact with the skin at a frequency of once a day to once 7 days.

8. A risperidone-containing transdermal adhesive patch comprising a support and a drug-containing layer formed on at least one surface of the support, wherein the drug-containing layer is composed of the preparation according to claim 6.

9. The adhesive patch according to claim 8, wherein an area of the drug-containing layer to be in contact with skin is 5 to 100 cm².

10. The adhesive patch according to claim 8, wherein a thickness of the drug-containing layer is 50 to 200 μm.
